(19) United States
(12) Patent Application Publication
(10) Pub. No.: US 2026/0283651 A1
(43) Pub. Date: Sep. 24, 2026

(54) DEVICES AND SYSTEMS FOR TREATING SPINAL DISORDERS

(71) Applicant: Warsaw Orthopedic, Inc., Warsaw, IN (US)

(72) Inventors: William A. Rezach, Covington, TN (US); Larry T. McBride, Jr., Germantown, TN (US); Joshua W. Simpson, Collierville, TN (US); Anna J. Krum, Southaven, MS (US); Joel P. Bales, Hernando, MS (US)

(21) Appl. No.: 19/474,133

(22) PCT Filed: Apr. 12, 2024
(86) PCT No.: PCT/US2024/024330

§ 371 Date: Oct. 9, 2025

Related U.S. Application Data
(60) Provisional application No. 63/459,083, filed on Apr. 13, 2023.
(60) Provisional application No. 63/597,034, filed on Nov. 8, 2023.

(51) Int. Cl. A61B 17/70

(52) U.S. Cl. CPC A61B 17/7034; A61B 17/7002; A61B 17/7037

(57) ABSTRACT

A receiver of a spinal screw assembly of a spinal stabilization system includes a shell and a bushing. A spinal screw assembly of a spinal stabilization system includes a screw and the receiver. A spinal stabilization system includes the screw, the receiver, and a rod. The bushing is configured to inhibit contact between the shell and the screw, the shell and the rod, and the screw and the rod. The bushing is formed from a material(s) different from the material(s) forming the screw, the shell and/or the rod.

### CROSS-REFERENCE TO RELATED APPLICATIONS

This application claims the benefit of, and priority to, U.S. Provisional Patent Application No. 63/459,083, filed on Apr. 13, 2023, and U.S. Provisional Patent Application No. 63/597,034, filed on Nov. 8, 2023. The entire contents of each of these applications is hereby incorporated herein by reference.

### FIELD

The present disclosure relates to spinal surgery and, more particularly, to devices and systems for treating disorders of the spine.

### BACKGROUND

Spinal disorders such as scoliosis and other curvature abnormalities, kyphosis, degenerative disc disease, disc herniation, osteoporosis, spondylolisthesis, stenosis, tumors, and fracture may result from trauma, disease, and/or degenerative conditions and may cause patient pain, deformity, nerve damage, and/or loss of mobility.

Surgical treatment of such spinal disorders includes correction, fusion, fixation, discectomy, laminectomy and/or implantable prosthetics. With respect to correction, for example, surgical treatment may include implantation of a system of rods and screws to provide spinal stabilization and maintain spinal curve correction.

While surgical treatments for spinal correction are generally effective, spinal correction is challenging in children due to the need to account for skeletal growth, while maintaining the corrections over time and minimizing the need for repeat surgeries until skeletal maturity.

### SUMMARY

As used herein, the term “distal” refers to the portion that is described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations and tolerances, up to and including plus or minus 10 percent. Further, any or all of the aspects described herein, to the extent consistent, may be used in conjunction with any or all of the other aspects described herein.

Provided in accordance with aspects of the present disclosure is a receiver configured to operably couple a spinal screw with a spinal rod. The receiver includes a shell and a bushing. The shell defines an internal cavity, a proximal opening in communication with the internal cavity, a distal opening in communication with the internal cavity, and first and second lateral openings in communication with the internal cavity. The proximal opening is configured to receive a screw distally therethrough (e.g., passing through proximal opening in a distal direction) with a head of the screw disposed within the internal cavity and a shank of the screw extending distally through the distal opening. The first and second lateral openings are configured to receive a rod extending through the internal cavity and laterally from opposing sides of the shell. The shell is formed from a first material. The bushing lines the first lateral opening, the second lateral opening, and at least a portion of the internal cavity. The bushing is formed from a second material different from the first material and is configured to inhibit contact between the shell and the screw, the shell and the rod, and the screw and the rod.

In an aspect of the present disclosure, the first material is a metal, and the second material is a plastic. Additionally or alternatively, in aspects, the first material is a relatively higher friction material and the second material is a relatively lower friction material exhibiting improved wear properties compared to the first material. In aspects, friction is measured in roughness (Ra), e.g., wherein the first material has a first Ra and the second material has a second Ra less than the first Ra.

In another aspect of the present disclosure, the bushing includes a bushing body that lines the at least a portion of the internal cavity, a first ring that lines the first lateral opening, and a second ring that lines the second lateral opening.

In yet another aspect of the present disclosure, the shell includes a first inwardly angled ramp adjacent to the distal opening and the bushing includes a second inwardly angled ramp lining the first inwardly angled ramp. The second inwardly angled ramp inhibits contact between the screw and the shell (e.g., the portion of the shell defining the distal opening) without extending into the distal opening of the shell.

In still another aspect of the present disclosure, the bushing includes an annular rib protruding radially inwardly into an interior of the bushing. The annular rib is configured to releasably retain the head of the screw within the internal cavity of the shell in spaced relation relative to the rod.

In still yet another aspect of the present disclosure, the bushing is configured to permit sliding of the rod relative to the bushing and the shell and/or angulation of the screw relative to the bushing and the shell.

Also provided in accordance with aspects of the present disclosure is a spinal screw assembly configured to operably couple a spinal screw with a spinal rod. The assembly includes a receiver and screw including a head and a shank extending from the head. The receiver includes a shell and a bushing. The shell defines an internal cavity having a screw head-receiving portion and a rod-receiving portion, a proximal opening in communication with the internal cavity, a distal opening in communication with the internal cavity, and first and second lateral openings in communication with the internal cavity. The proximal opening is configured to receive the screw distally therethrough with the head of the screw disposed within the screw head-receiving portion of the internal cavity and the shank of the screw extending distally through the distal opening. The first and second lateral openings are configured to receive a rod extending through the rod-receiving portion of the internal cavity. The shell is formed from a first material. The bushing lines at least a portion of the internal cavity and is formed from a second material different from the first material. The bushing is configured to inhibit contact between the shell and the screw and to maintain a gap between the screw and the rod within the internal cavity, thereby inhibiting contact between the screw and the rod within the internal cavity.

In an aspect of the present disclosure, the bushing lines the first and second lateral openings and is configured to inhibit contact between the shell and the rod.

In another aspect of the present disclosure, the bushing includes an annular rib protruding radially inwardly into an interior of the bushing. The annular rib is configured to releasably retain the head of the screw within the screw head-receiving portion of the internal cavity and to maintain the gap.

In another aspect of the present disclosure, the receiver is configured to permit angulation of the screw relative to the receiver.

In still another aspect of the present disclosure, the first material is a metal, and the second material is a plastic. Additionally or alternatively, in aspects, the first material is a relatively higher friction material (higher Ra) and the second material is a relatively lower friction material (lower Ra) exhibiting improved wear properties compared to the first material.

In yet another aspect of the present disclosure, the bushing includes a bushing body that lines the at least a portion of the internal cavity, a first ring that lines the first lateral opening, and a second ring that lines the second lateral opening.

In still yet another aspect of the present disclosure, the shell includes a first inwardly angled ramp adjacent to the distal opening and the bushing includes a second inwardly angled ramp lining the first inwardly angled ramp and inhibiting contact between the screw and the shell (e.g., the portion of the shell defining the distal opening) without extending into the distal opening of the shell.

A spinal stabilization system provided in accordance with aspects of the present disclosure includes a rod, a screw including a head and a shank extending from the head, and a receiver. The receiver includes a shell and a bushing. The shell defines an internal cavity having a screw head-receiving portion and a rod-receiving portion, a proximal opening in communication with the internal cavity, a distal opening in communication with the internal cavity, and first and second lateral openings in communication with the internal cavity. The proximal opening is configured to receive the screw distally therethrough with the head of the screw disposed within the screw head-receiving portion and the shank of the screw extending distally through the distal opening. The first and second lateral openings are configured to receive the rod extending through the internal cavity. The bushing lines at least a portion of the interior surface of the shell and the first and second lateral openings. The bushing is configured to permit sliding of the rod relative to the bushing and the shell, permit angulation of the screw relative to the bushing and the shell, inhibit contact between the shell and the screw, inhibit contact between the shell and the rod, and maintain a gap between the screw and the rod within the internal cavity.

In an aspect of the present disclosure, the bushing includes a bushing body that lines the at least a portion of the internal cavity, a first ring that lines the first lateral opening, and a second ring that lines the second lateral opening.

In another aspect of the present disclosure, the bushing includes an annular rib protruding radially inwardly into an interior of the bushing. The annular rib is configured to releasably retain the head of the screw within the internal cavity and to maintain the gap between the screw and the rod within the internal cavity.

In still another aspect of the present disclosure, the shell is formed from a first material and the bushing is formed from a second material different from the first material. In such aspects, the first material may be a metal and/or the second material may be a plastic. Additionally or alternatively, in aspects, the first material is a relatively higher friction material (higher Ra) and the second material is a relatively lower friction material (lower Ra) exhibiting improved wear properties compared to the first material.

In yet another aspect of the present disclosure, the bushing fully surrounds the rod within each of the first and second lateral openings of the shell.

In still yet another aspect of the present disclosure, the bushing includes an inwardly angled ramp configured to inhibit contact between the screw and the shell (e.g., the portion of the shell defining the distal opening) without extending into the distal opening of the shell.

Another receiver provided in accordance with the present disclosure and configured to operably couple a spinal screw with a spinal rod includes a shell, a bushing, and an end cap. The shell includes a base and first and second opposing side walls extending upwardly from the base. The shell defines a rod receiving passageway extending transversely through the shell. The bushing is disposed within the shell and includes a base and first and second opposing side walls extending upwardly from the base. The bushing defines a rod receiving passageway extending transversely through the bushing. The rod receiving passageways of the shell and the bushing are aligned and configured to slidably receive a rod therethrough with the bushing supporting the rod to inhibit contact between the rod and the shell. The end cap is engaged to the base of the shell to retain the bushing within the shell.

In an aspect of the present disclosure, the shell is formed from a first material and the bushing is formed from a second material different from the first material.

In another aspect of the present disclosure, the bushing and the shell define complementary anti-rotation surfaces configured to maintain the rod receiving passageways of the shell and the bushing in alignment with one another.

In still another aspect of the present disclosure, the bushing and the shell engage one another to inhibit upward movement of the bushing within the shell and the bushing and the end cap engage one another to inhibit downward movement of the bushing within the shell.

In yet another aspect of the present disclosure, the end cap is at least one of threadingly engaged with the base of the shell or welded to the base of the shell.

In still yet another aspect of the present disclosure, a set screw is configured to engage the first and second opposing side walls of the shell to retain the rod within the rod receiving passageways of the shell and the bushing. The set screw may include a body and a bushing disc engaged with the body and configured to inhibit contact between the body of the set screw and the rod.

In an aspect of the present disclosure, the bushing includes an annular rib protruding radially inwardly into an interior of the bushing. The annular rib is configured to releasably retain a head of a screw within an internal cavity of the bushing in spaced relation relative to the rod.

Another spinal screw assembly provided in accordance with the present disclosure includes a screw including a head and a shank extending from the head and a receiver according to any of the aspects detailed above or otherwise herein.

Another spinal stabilization system provided in accordance with the present disclosure includes a rod, a screw including a head and a shank extending from the head, and a receiver. The receiver includes a shell according to any of the aspects detailed above or otherwise herein and a bushing disposed within the shell and configured according to any of the aspects detailed above or otherwise herein. The rod is configured for slidable receipt within the rod receiving passageways of the shell and the bushing with the rod supported by the bushing to inhibit contact between the rod and the shell.

In an aspect of the present disclosure, the receiver further includes a set screw configured to engage the first and second opposing side walls of the shell to retain the rod within the rod receiving passageways of the shell and the bushing. In such aspects, the set screw may include a body and a bushing disc engaged with the body and configured to inhibit contact between the body of the set screw and the rod.

In another aspect of the present disclosure, in a fully engaged position of the set screw, a gap is defined between the bushing disc and the rod to enable sliding of the rod through the rod receiving passageways of the shell and the bushing. The fully engaged position may be defined by contact between a collar of the set screw and free ends of the first and second side walls of the shell.

In another aspect of the present disclosure, the bushing is configured to maintain a gap between the rod and the head of the screw.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 shows a system for treating disorders of the spine in accordance with the present disclosure installed on a patient's spine;

FIG. 2 is a perspective view of a screw assembly provided in accordance with the present disclosure, configured for use with the system of FIG. 1, and including a screw and a receiver;

FIG. 3 is an exploded, perspective view of the screw assembly of FIG. 2;

FIG. 4 is a perspective view of the receiver of the screw assembly of FIG. 2;

FIG. 5 is an exploded, perspective view of the receiver of FIG. 4;

FIG. 6A is a transverse, cross-sectional view of the receiver of FIG. 4 taken across section line “6A-6A” of FIG. 4;

FIG. 6B is a transverse, cross-sectional view of the receiver of FIG. 4 taken across section line “6B-6B” of FIG. 4;

FIG. 7 is a transverse, cross-sectional view of the screw assembly of FIG. 2 illustrating insertion of the screw into the receiver;

FIG. 8 is a transverse, cross-sectional view of the screw assembly of FIG. 2 illustrating the screw engaged within the receiver;

FIG. 9 is a transverse, cross-sectional view of the screw assembly of FIG. 2 illustrating the screw engaged within the receiver and a rod extending through the receiver;

FIG. 10 is a transverse, cross-sectional view of the screw assembly of FIG. 2 illustrating the screw engaged within the receiver and the rod extending through the receiver, wherein the screw is disposed in an angled orientation relative to the receiver;

FIG. 11 is a perspective view of another screw assembly provided in accordance with the present disclosure, configured for use with the system of FIG. 1, and including a screw, a receiver, and a cap and including a rod extending therethrough;

FIGS. 12 and 13 are side and transverse, cross-sectional views, respectively, of the screw assembly of FIG. 11 including the rod extending therethrough;

FIGS. 14 and 15 are transverse, cross-sectional and exploded views, respectively, of the screw assembly of FIG. 11;

FIG. 16 is a perspective view of a shell of the receiver of the screw assembly of FIG. 11;

FIG. 17 is a perspective view of a bushing of the receiver of the screw assembly of FIG. 11;

FIG. 18 is a bottom perspective view of the cap of the receiver of the screw assembly of FIG. 11; and

FIGS. 19 and 20 are transverse cross-sectional views of the screw assembly of FIG. 11 illustrating insertion of the screw into the receiver and engagement of the screw assembly within the receiver, respectively.

### DETAILED DESCRIPTION

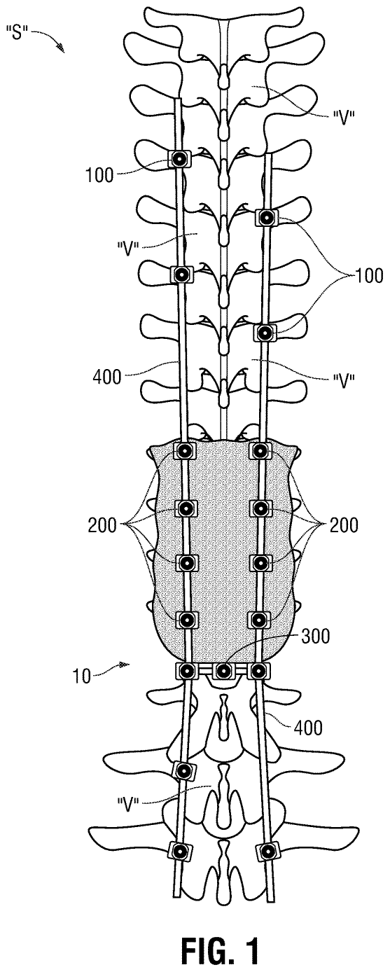

Referring to FIG. 1, a system for treating disorders of the spine in accordance with the present disclosure is generally identified by reference numeral 10 and shown installed on a patient's spine “S.” System 10 includes one or more polyaxial screw assemblies 100, one or more fixed angle screw assemblies 200, one or more connectors 300, and one or more rods 400. Each of the screw assemblies 100, 200 is anchored in a vertebrae “V” of the patient's spine “S.” Each of the one or more connectors 300 connects a screw assembly 100, 200 or a rod 400 with another screw assembly 100, 200 or rod 400. Each rod 400 is received within one or more screw assemblies 100, 200 and generally extends along at least a portion of a length of the patient's spine “S.”

Although screw assemblies 100, 200 are shown anchored in pedicle bone, it is also contemplated that screw assemblies 100, 200 can be anchored in cortical bone, or in any other suitable bone, whether vertebral or otherwise. Further, although polyaxial screw assemblies 100 and fixed angle screw assemblies 200 are shown in certain locations, it is understood that polyaxial screw assemblies 100, fixed angle screw assemblies 200, and/or any other suitable screw assemblies may be used, in any suitable location. For example and without limitation, uniaxial screw assemblies, e.g., confining movement of the screw to within one plane, are also contemplated in accordance with the present disclosure. As another example without limitation, the screw assemblies can be installed at a sagittal trajectory, a transverse trajectory, and/or at any suitable location.

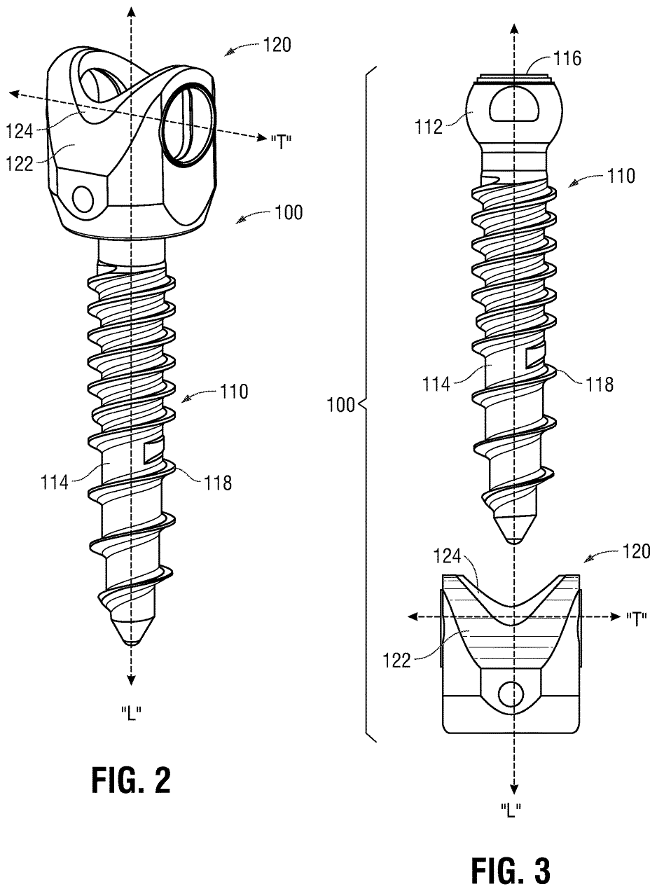

Turning to FIGS. 2 and 3, a polyaxial screw assembly 100 is provided in accordance with the present disclosure and configured for use with system 10 (FIG. 1) or any other suitable system for treating disorders of the spine. Although described herein with reference to polyaxial screw assembly 100, the aspects and features of the present disclosure, to the extent consistent, are also applicable for use with other screw assemblies such as, for example, fixed angle screw assemblies (e.g., fixed angle screw assemblies 200 (FIG. 1)), monoaxial screw assemblies, etc.

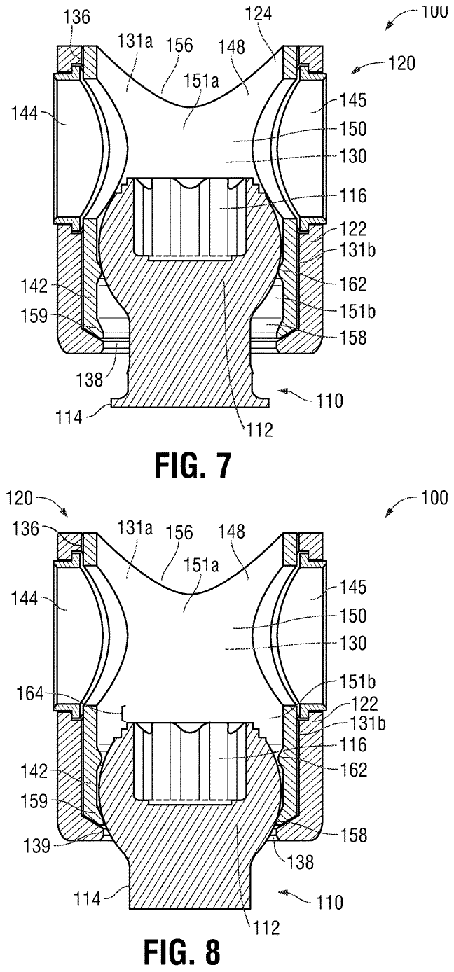

Screw assembly 100 includes a screw 110 and a receiver 120. Screw 110 includes a head 112 defining a generally spherical configuration and a shank 114 extending distally from head 112. Head 112 includes a socket 116 (FIGS. 7 and 8) defined therein opposite shank 114. Socket 116 may define a star-shaped configuration or any other suitable configuration for at least partial complementary receipt of a driving tool (not shown) for rotationally driving shank 114 of screw 110 into tissue, e.g., vertebral bone. Shank 114 includes helical threading 118 to facilitate driving of shank 114 into and retention of shank 114 within tissue, e.g., vertebral bone, although other configurations are also contemplated. Screw 110 may be formed from a metallic material such as, for example, titanium or cobalt chrome. Other suitable metallic materials forming screw 110 such as, for example, stainless steel, are also contemplated, as are configurations wherein screw 110 is formed from multiple materials, e.g., wherein head 112 is formed from a first material and wherein shank 114 is formed from a second, different material.

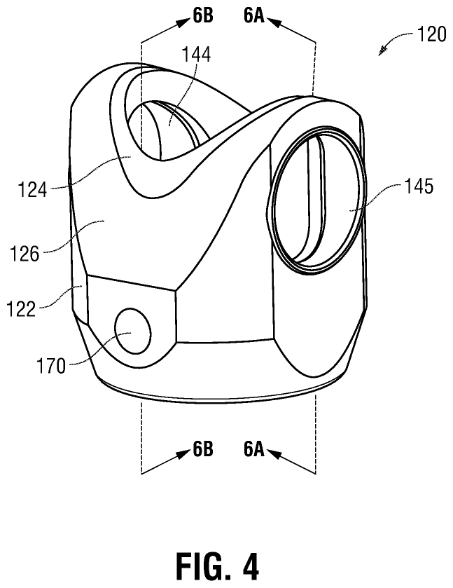

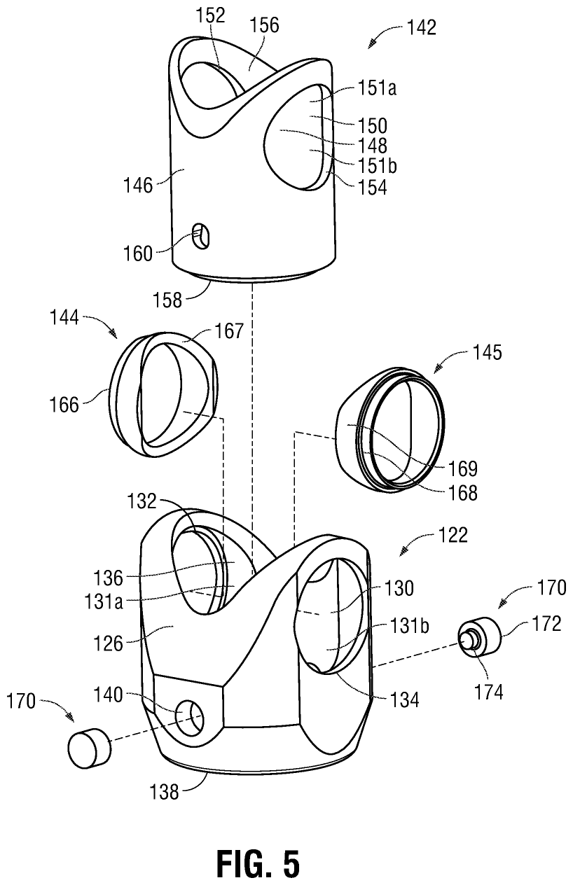

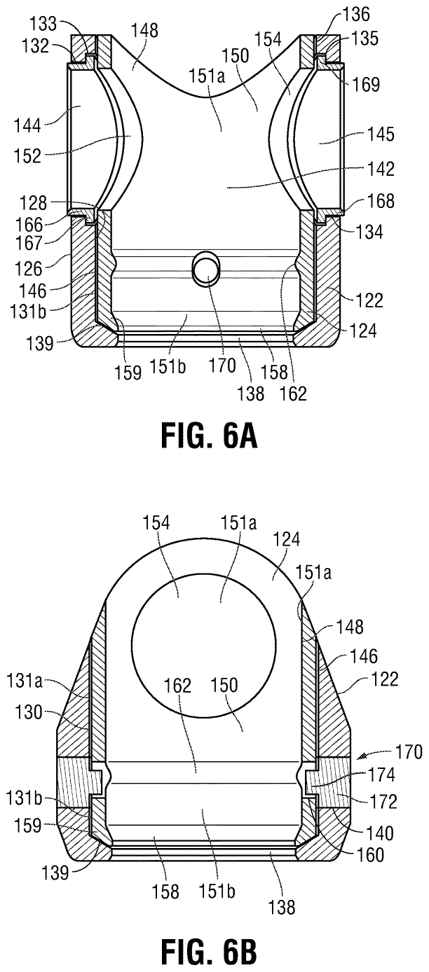

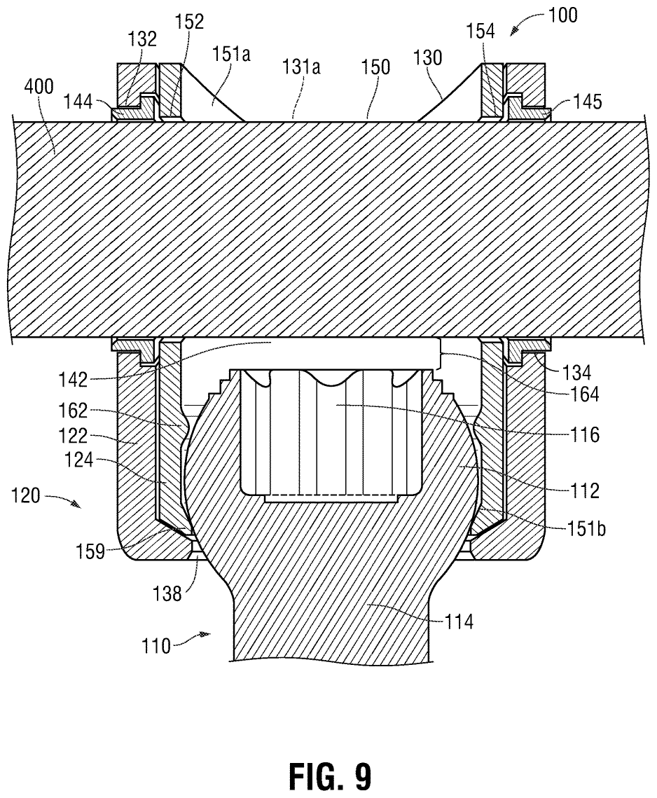

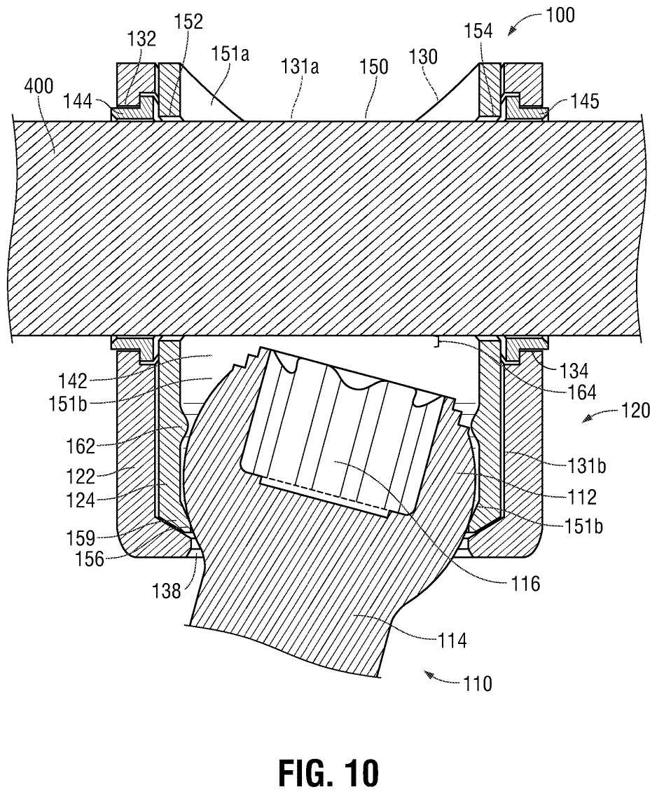

With additional reference to FIGS. 4-6B, receiver 120 includes a shell 122 and a bushing 124. Shell 122 is configured to provide strength and structural support to receiver 120 and may be formed from a metallic material such as, for example, titanium or cobalt chrome. Other suitable metallic materials forming shell 122 such as, for example, stainless steel, are also contemplated. Bushing 124 may be formed from a polymeric material such as, for example, polyether ether ketone (PEEK) or ultra-high-molecular-weight polyethylene (UHMWPE). Other suitable materials include, without limitation, polyethylene, carbon fiber reinforced PEEK, carbon composite, polylactic acid (PLA), or polyglycolic acid (PGA). In configurations where bushing 124 is formed from multiple components, two or more of the components may be formed from similar or different polymeric materials. As detailed below, bushing 124 is configured to inhibit contact between shell 122 and screw 110, between shell 122 and a rod 400 (FIGS. 1 and 9) received through shell 122, and between screw 110 and the rod 400 (FIGS. 1 and 9). Thus, by inhibiting contact between these metallic components, bushing 124 inhibits metal fretting and debris generation from metal-on-metal contact, particularly with respect to relative movement between metal components such as, for example, sliding of rod 400 (FIGS. 1 and 9) through shell 122 and/or angulation of screw 110 relative to shell 122 (see FIG. 10). Bushing 124, as also detailed below, is further configured to facilitate operable engagement of head 112 of screw 110 with receiver 120 to retain head 112 of screw 110 within receiver 120 while permitting polyaxial angulation of head 112 of screw 110 relative to receiver 120. In aspects, the material or materials forming shell 122 are relatively higher friction material(s) (higher roughness (Ra)) while the material or materials forming bushing 124 are relatively lower friction material(s) (lower Ra), thus exhibiting improved wear properties compared to the first material(s).

Shell 122 of receiver 120 includes an exterior surface 126 and an interior surface 128 that defines an interior cavity 130 within shell 122. Interior cavity 130 includes a rod-receiving portion 131a and a screw head-receiving portion 131b. Shell 122 defines a closed, vest-shaped configuration including first and second lateral arm openings 132, 134, a proximal neck opening 136, and a distal waist opening 138. First and second lateral arm openings 132, 134 are transversely aligned with one another, connected within shell 122 via rod-receiving portion 131a of interior cavity 130, and configured to slidably receive a rod 400 (FIGS. 1 and 9) to enable rod 400 (FIGS. 1 and 9) to extend transversely through first lateral arm opening 132, rod-receiving portion 131a of interior cavity 130, and second lateral arm opening 134 such that receiver 120 slidably supports rod 400 (FIGS. 1 and 9) therethrough. Shell 122 defines first and second annular shoulders 133, 135 surrounding first and second lateral arm openings 132, 134, respectively, such that first and second lateral arm openings 132, 134 include inner portions defining first diameters and outer portions defining second diameters smaller than the first diameters.

Proximal neck opening 136 of shell 122 of receiver 120 is configured to receive screw 110 therethrough, while distal waist opening 138 of shell 122 of receiver 120 is configured to permit passage of shank 114 of screw 110 therethrough but inhibit passage of head 112 of screw 110 therethrough. More specifically, distal waist opening 138 of shell 122 defines a reduced diameter as compared to screw head-receiving portion 131b of interior cavity 130 of receiver 120 such that the full diameter portion of head 112 of screw 110 is insertable into head-receiving portion 131b of interior cavity 130 but inhibited from passing from head-receiving portion 131b of interior cavity 130 through distal waist opening 138 of shell 122 of receiver 120. In aspects, shell 122 defines an inwardly angled ramp 139 adjacent distal waist opening 138 to define a smooth or smoother transition between the relatively larger diameter screw head-receiving portion 131b of interior cavity 130 and the relatively smaller diameter of distal waist opening 138.

The above-detailed configuration of shell 122 enables insertion of screw 110, led by shank 114, through proximal neck opening 136 and rod-receiving portion 131a of interior cavity 130 to operably engage head 112 of screw 110 within screw head-receiving portion 131b of interior cavity 130 while shank 114 extends from screw head-receiving portion 131b of interior cavity 130 and through distal waist opening 138 such that shank 114 extends distally from receiver 120. Screw head-receiving portion 131b of interior cavity 130 defines a generally cylindrical configuration and extends substantially coaxially with a longitudinal axis “L” defined through proximal neck opening 136 and distal waist opening 138 of shell 122 and substantially perpendicularly relative to a transverse axis “T” defined through first and second lateral arm openings 132, 134 of shell 122.

Shell 122 of receiver 120 further defines one or more pin slots 140 extending transversely from exterior surface 126 to interior surface 128 to provide access to screw head-receiving portion 131b of interior cavity 130. In aspects, the one or more pin slots 140 includes a pair of diametrically opposed pin slots 140 oriented to define an axis that is substantially perpendicular relative to both the longitudinal axis “L” and the transverse axis “T.” As an alternative to the closed configuration of shell 122 detailed above, shell 122 may define an open configuration. That is, rather than circumferentially enclosed lateral arm openings 132, 134 requiring sliding of rod 400 (FIGS. 1 and 9) along a longitudinal axis of rod 400 (FIGS. 1 and 9) sequentially through one opening 132, 134, rod-receiving portion 131a of interior cavity 130, and the other opening 132, 134 to slidably support rod 400 (FIGS. 1 and 9) within receiver 120, shell 122 may define U-shaped arm openings 132, 134 and an open top (not shown) to enable rod 400 (FIGS. 1 and 9) to be “dropped” into openings 132, 134 and rod-receiving portion 131a in a direction substantially perpendicular to the longitudinal axis of rod 400 (FIGS. 1 and 9). In such configurations, a set screw, cap, or other suitable component (not shown) may be engaged with shell 122 to retain rod 400 (FIGS. 1 and 9) in slidable engagement in openings 132, 134 and rod-receiving portion 131a of shell 122.

Continuing with reference to FIGS. 2-6B, bushing 124 of receiver 120 is disposed within shell 122 and includes a bushing body 142 and first and second bushing rings 144, 145, respectively. Although described herein as three separate components (e.g., bushing body 142, first bushing ring 144, and second bushing ring 145), it is contemplated that bushing 124 may be formed from greater or fewer components and/or that the component(s) forming bushing 124 define other configurations. Further, although bushing 124 is detailed herein for mechanical engagement within shell 122, other suitable manners of securing bushing 124 within shell 122 are also contemplated such as, for example, adhesion, bonding, overmolding, spraying, depositing, etc.

Bushing body 142 defines a generally cylindrical shaped configuration and includes an exterior surface 146 and an interior surface 148 that defines a substantially cylindrical interior cavity 150 within bushing body 142. Interior cavity 150 defines a rod-receiving portion 151a and a screw head-receiving portion 151b. Bushing body 142 further defines first and second transverse openings 152, 154, a proximal end opening 156, and a distal end opening 158. First and second transverse openings 152, 154 are transversely aligned with one another along an axis that is substantially perpendicular to a longitudinal axis of bushing body 142 (and substantially coaxial with transverse axis “T” of receiver 120) while proximal and distal end openings 156, 158 are longitudinally aligned with one another along an axis that is substantially coaxial with the longitudinal axis of bushing body 142 (and substantially coaxial with longitudinal axis “L” of receiver 120).

Bushing body 142 is configured for insertion through proximal neck opening 136 of shell 122 and into interior cavity 130 of shell 122 such that: exterior surface 146 of bushing body 142 substantially abuts and lines interior surface 138 of shell 122; first and second transverse openings 152, 154 of bushing body 142 align with respective first and second arm openings 132, 134 of shell 122; proximal end opening 156 of bushing body 142 aligns with proximal neck opening 136 of shell 122; and distal end opening 158 of bushing body 142 aligns with distal waist opening 138 of shell 122. Bushing body 142 may further include one or more pin slots 160 extending transversely from exterior surface 146 to interior surface 148 to provide access to screw head-receiving portion 151b of interior cavity 150. In aspects, the one or more pin slots 160 includes a pair of diametrically opposed pin slots 160, similarly as detailed above with respect to pin slots 140 of shell 122. The one or more pin slots 160 of bushing body 142 may each define a diameter less than a diameter defined by each of pin slots 140 of shell 122.

With bushing body 142 received within interior cavity 130 of shell 122, a proximal end portion of bushing body 142 extends at least partially into proximal neck opening 136 of shell 122 to line the interior annular surface of shell 122 that defines proximal neck opening 136. Further, with bushing body 142 received within interior cavity 130 of shell 122, an inwardly angled ramp 159 of bushing body 142 lines inwardly angled ramp 139 of shell 122. Inwardly angled ramp 159 also defines a reduced diameter at distal end opening 158 of bushing body 142 as compared to screw head-receiving portion 151b of interior cavity 150 such that the full diameter portion of head 112 of screw 110 is insertable into head-receiving portion 151b of interior cavity 150 of bushing body 142 but inhibited from passing from head-receiving portion 151b through distal end opening 158 of bushing body 142. In aspects, bushing body 142 does not extend into distal waist opening 138 of shell 122. Despite bushing body 142 not extending into distal waist opening 138, bushing body 142 inhibits contact between screw 110 and the interior annular surface of shell 122 that defines distal waist opening 138 even during angulation of screw 110 relative to receiver 120.

Interior cavity 150 of bushing body 142, as noted above, defines a rod-receiving portion 151a and a screw head-receiving portion 151b. Rod-receiving portion 151a of interior cavity 150 of bushing body 142 is disposed towards a proximal end portion of bushing body 142 and defines a transverse passage extending between first and second transverse openings 152, 154 to enable slidable receipt of a rod 400 (FIGS. 1 and 9) through rod-receiving portion 151a of interior cavity 150 of bushing body 142.

Screw head-receiving portion 151b of interior cavity 150 of bushing body 142 extends longitudinally from rod-receiving portion 151a of interior cavity 150 to inwardly angled ramp 159 of bushing body 142. An internal annular rib 162 protrudes inwardly from interior surface 148 of bushing body 142 into screw head-receiving portion 151b of interior cavity 150. Annular rib 162 is longitudinally spaced from inwardly angled ramp 159 a sufficient distance to enable capture of the full diameter portion of head 112 of screw 110 therebetween to substantially inhibit longitudinal movement of head 112 of screw 110 relative to receiver 120 while permitting angulation of head 112 of screw 110 relative to receiver 120. As detailed below, annular rib 162 permits positioning of head 112 of screw 110 within screw head-receiving portion 151b of interior cavity 150 of bushing body 142 between annular rib 162 and inwardly angled ramp 159 and, once head 112 is received therein, helps inhibit head 112 from backing out of screw head-receiving portion 151b of interior cavity 150. Further, annular rib 162 is longitudinally spaced from rod-receiving portion 151a of interior cavity 150 to define a gap 164 between head 112 of screw 110 and rod 400 (FIGS. 9 and 10), thus inhibiting contact between head 112 of screw 110 and rod 400 regardless of the angular orientation of screw 110 relative to receiver 120 (see FIGS. 9 and 10).

Referring still to FIGS. 2-6B, first and second bushing rings 144, 145 each includes a ring body 166, 168 and a ring flange 167, 169, respectively. Ring bodies 166, 168 are configured for insertion, from interior cavity 130 of shell 122, into first and second arm openings 132, 134, respectively, of shell 122 to line the respective interior annular surfaces of shell 122 that define arm openings 132, 134. Ring flanges 167, 169 extend radially outwardly from respective ring bodies 166, 168 and are configured for receipt within first and second annular shoulders 133, 135, respectively, of shell 122 to inhibit passage of first and second bushing rings 144, 145 through first and second arm openings 132, 134, respectively. First and second bushing rings 144, 145 are configured for positioning in the above-detailed manner prior to insertion of bushing body 142 into internal cavity 130 of shell 122 such that, upon subsequent positioning of bushing body 142 within internal cavity 130 of shell 122, bushing body 142 inhibits first and second bushing rings 144, 145 from backing out of first and second arm openings 132, 134, thereby retaining first and second bushing rings 144, 145 in position.

With first and second bushing rings 144, 145 and bushing body 142 positioned within internal cavity 130 of shell 122 as detailed above, one or more pins 170 (each corresponding to one pair of aligned pin slots 140, 160 of shell 122 and bushing body 142 of bushing 124, respectively), may be installed to secure bushing 124 within shell 122. Each pin 170 includes a relatively larger diameter head 172 and a relatively smaller diameter shaft 174. Each pin 170 is insertable, led by shaft 174 and from the exterior of shell 122, through the aligned pin slots 140, 160 of shell 122 and bushing body 142, respectively. The relatively smaller diameter shaft 174 is insertable through both pin slots 140, 160 of shell 122 and bushing body 142, while the relatively larger diameter head 172 is inhibited from passing into pin slot 160 of bushing body 142 and, thus, is seated within pin slot 140 of shell 122. Once positioned in this manner, each pin 170 may be secured in position, e.g., via welding head 172 to shell 122. Thus, bushing 124 is secured in position within and relative to shell 122. Pins 170 also maintains alignment of the passageways through bushing rings 144, 145 of bushing 124 with the respective lateral arm openings 132, 134 defined through shell 122.

Referring to FIGS. 7-10, assembly and use of screw assembly 100 is detailed. With initial reference to FIGS. 7-8, screw 110, led by shank 114, is inserted through proximal end opening 156 of bushing body 142 (and, thus, proximal neck opening 136 of shell 122), traversing rod-receiving portion 151a of interior cavity 150 (and, thus, rod-receiving portion 131a of interior cavity 130), and into screw head-receiving portion 151b of interior cavity 150 of bushing body 142 (and, thus, screw head-receiving portion 131b of interior cavity 130), such that shank 114 of screw 110 extends distally through distal end opening 158 of bushing body 142 (and, thus, distal waist opening 138 of shell 122) while head 112 of screw 110 is retained within screw head-receiving portion 151b of interior cavity 150 of bushing body 142 (and, thus, screw head-receiving portion 131b of interior cavity 130 of shell 122). More specifically, as head 112 of screw 110 is urged longitudinally through interior cavity 150, head 112 contacts annular rib 162 and compresses annular rib 162 at least partially into bushing body 142 to enable passage of the full diameter portion of head 112 through the reduced-diameter space defined by annular rib 162 and into position between annular rib 162 and inwardly angled ramp 159 of bushing body 142. Once the full diameter portion of head 112 traverses annular rib 162, annular rib 162 resiliently returns to its at-rest configuration to thereby capture head 112 between annular rib 162 and angled ramp 159 of bushing body 142 while still permitting angulation of screw 110 relative to receiver 120. Annular rib 162, more specifically, maintains gap 164 between head 112 of screw 110 and rod 400 (FIGS. 9 and 10), thus inhibiting contact therebetween regardless of the angulation of screw 110 relative to receiver 120 and rod 400 (FIGS. 9 and 10).

Turning to FIG. 9, once screw 110 is operably coupled to receiver 120 as detailed above, screw 110 may be anchored within tissue, e.g., vertebral bone, at a target location. The above process may be repeated to install plural screw assemblies 100 at various locations, e.g., along a patient's spine “S” (see FIG. 1). Once the desired screw assemblies 100 are installed, one or more rods 400 may be slidably received through one or more of the screw assemblies 100. More specifically, to slidably engage a rod 400 with a receiver 120 of a screw assembly 100, rod 400 is inserted, along a longitudinal axis thereof, through one of the first or second bushing rings 144, 145 of bushing 124 (and, thus, through one of the first or second lateral arm openings 132, 134 of shell 122), through one of the first or second transverse openings 152, 154 of bushing body 142, through rod-receiving portion 151a of interior cavity 150 of bushing body 142 (and, thus, through rod-receiving portion 131a of interior cavity 130 of shell 122), through the other of the first or second transverse openings 152, 154 of bushing body 142, and through the other of the first or second bushing rings 144, 145 of bushing 124 (and, thus, through the other of the first or second lateral arm openings 132, 134 of shell 122).

Referring to FIG. 10, with screw 110 and rod 400 operably engaged with receiver 120 as detailed above, metal-to-metal contact between shell 122 and screw 110, shell 122 and rod 400, or screw 110 and rod 400 are inhibited, while rod 400 is permitted to slide through and relative to receiver 120 and angulation of screw 110 relative to receiver 120 is also permitted. Thus, for example, sliding of rod 400 and angulation of screw 110 are enabled, e.g., to adapt to growth of a child patient, while metal fretting and debris generation from metal-on-metal contact are inhibited.

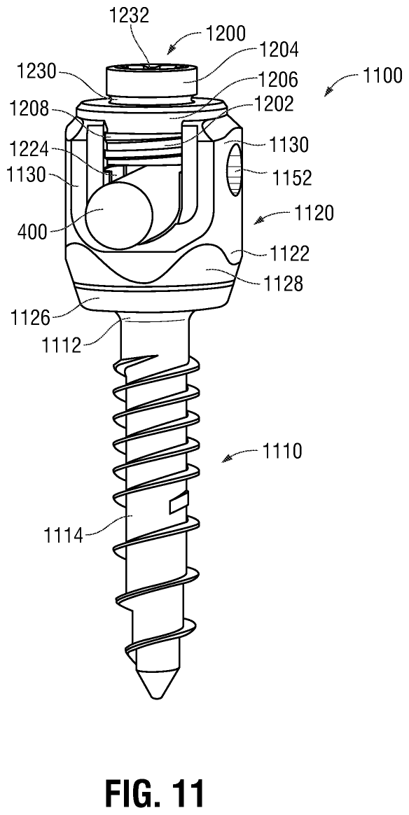

Turning to FIGS. 11-13, another polyaxial screw assembly 1100 is provided in accordance with the present disclosure and configured for use with system 10 (FIG. 1) or any other suitable system for treating disorders of the spine. Although described as a polyaxial screw assembly 1100, the aspects and features of screw assembly 1100, to the extent consistent, are also applicable for use with other screw assemblies such as, for example, fixed angle screw assemblies (e.g., fixed angle screw assemblies 200 (FIG. 1)), monoaxial screw assemblies, etc. Further, to the extent consistent, any of the aspects and features of screw assembly 100 (FIGS. 2 and 3) detailed above may be utilized with screw assembly 1100 and vice versa. Accordingly, differences between screw assembly 1100 and screw assembly 100 (FIGS. 2 and 3) are described in detail below while similarities are summarily described or omitted entirely.

Screw assembly 1100 includes a screw 1110, a receiver 1120, and a set screw 1200. Screw 1110 includes a head 1112 and a shank 1114 and may include any of the features of screw 110 (FIGS. 2 and 3) and/or any other suitable screw.

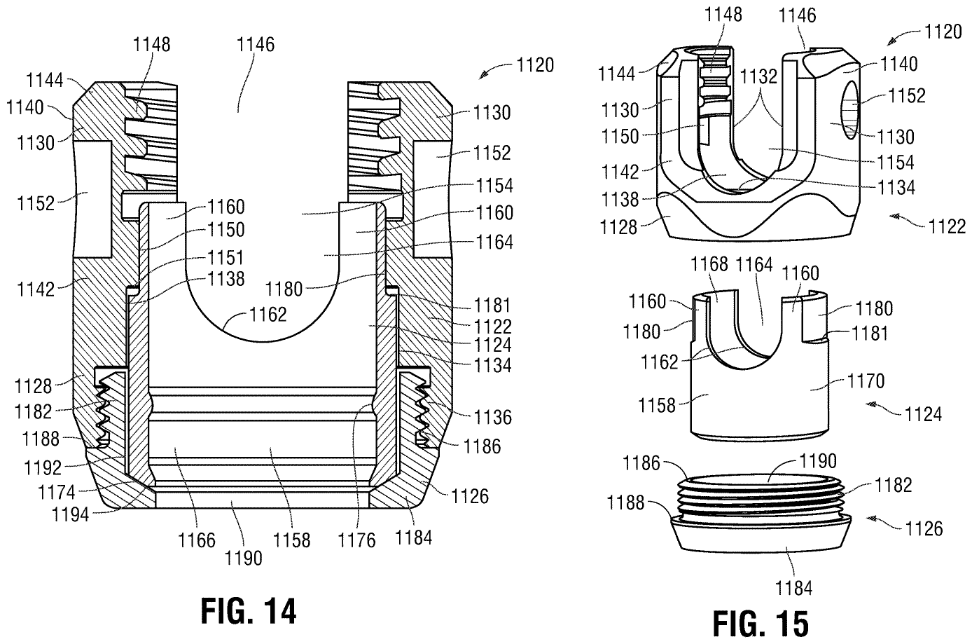

With additional reference to FIGS. 14-17, receiver 1120 includes a shell 1122, a bushing 1124, and an end cap 1126. Shell 1122 is configured to provide strength and structural support to receiver 1120 and may be formed from any of the materials detailed above with respect to shell 122 (FIGS. 4-6B) or any other suitable materials. Bushing 1124 is configured to inhibit contact between metal components and/or high friction components, e.g., shell 1122 and screw 1110 and/or shell 1122 and rod 400 (FIGS. 11-13), to thereby inhibit fretting and debris generation, particularly with respect to relative movement between these components such as, for example, sliding of rod 400 (FIGS. 1-13) through shell 1122, tilting of rod 400 (FIGS. 1-13) relative to shell 1122, and/or angulation of screw 1110 relative to shell 1122. Bushing 1124, as also detailed below, is further configured to facilitate operable engagement of head 1112 of screw 1110 within receiver 1120. Bushing 1124 may be formed from any of the materials detailed above with respect to bushing 124 (FIGS. 4-6B). End cap 1126 is configured to retain shell 1122 and bushing 1124 in engagement with one another and may be formed from any of the materials detailed above with respect to shell 122 (FIGS. 4-6B) or any other suitable materials. In particular, end cap 1126 may be formed from the same material as shell 1122 or a different material.

Continuing with reference to FIGS. 14-16, shell 1122 of receiver 1120 includes a base 1128 and first and second opposing side walls 1130 extending upwardly from base 1128 in spaced relation relative to one another to define first and second opposing saddles 1132. Base 1128 defines a generally cylindrical internal cavity 1134 open to the bottom end of base 1128 and includes internal threading 1136 surrounding internal cavity 1134. The exterior of base 1128 may be generally cylindrical, although other configurations are also contemplated such as, for example, polygonal configurations and/or configurations including combinations of flat and arcuate sides. A portion of the exterior surface of base 1128 disposed towards the bottom end of base 1128 may taper radially inwardly, as shown, although other configurations are also contemplated.

Side walls 1130 of shell 1122 include an interior surface 1138, an exterior surface 1140, a bottom end 1142 connected to base 1128, and a free upper end 1144. Interior surfaces 1138 of side walls 1130 are arcuate at least at free upper ends 1144 of side walls 1130 such that free upper ends 1144 of side walls 1130 cooperate to partially define a circular upper opening 1146 of shell 1122. Further, interior surfaces 1138 of side walls 1130 include arcuate threaded portions 1148 positioned towards free upper ends 1144 of side walls 1130 and extending about opposing portions of circular upper opening 1146. Arcuate threaded portions 1148 are configured to receive set screw 1200 of screw assembly 1100 in threaded engagement, as detailed below, to thereby retain rod 400 (FIGS. 11-13) within receiver 1120.

Interior surfaces 1138 of side walls 1130 also include opposed anti-rotation surfaces 1150 positioned towards bottom ends 1142 of side walls 1130. Anti-rotation surfaces 1150 may be configured as inwardly protruding flats (as shown), or any other suitable anti-rotation surfaces. As detailed below, anti-rotation surfaces 1150 cooperate with corresponding features of bushing 1124 to rotationally lock shell 1122 and bushing 1124 relative to one another, thus inhibiting relative rotation therebetween.

Exterior surfaces 1140 of side walls 1130 may include opposed engagement recesses 1152 defined therein. Engagement recesses 1152 are configured to receive suitable tools (not shown) to facilitate retaining and/or manipulating shell 1122 during assembly of screw assembly 1100, attachment of screw assembly 1100 to bone, and/or coupling of screw assembly 1100 with rod 400 (FIGS. 11-13).

Saddles 1132 of shell 1122, as noted above, are defined between side walls 1130. Saddles 1132 may define a U-shaped configuration or any other suitable configuration and are aligned with one another to define a U-shaped rod-receiving passageway 1154 extending transversely through shell 1122 of receiver 1120, e.g., for receiving rod 400 (FIGS. 11-13). U-shaped rod-receiving passageway 1154 communicates with both circular upper opening 1146 and generally cylindrical internal cavity 1132 within shell 1122.

Referring to FIGS. 14, 15, and 17, bushing 1124 of receiver 1120 includes a base 1158 and first and second opposing side walls 1160 extending upwardly from base 1158 in spaced relation relative to one another to define first and second opposing saddles 1162. Saddles 1162 of bushing 1124 may define a U-shaped configuration or any other suitable configuration and are aligned with one another to define a U-shaped rod-receiving passageway 1164 extending transversely through bushing 1124 of receiver 1120, e.g., for receiving rod 400 (FIGS. 11-13). Bushing 1124 further defines a generally cylindrical internal cavity 1166 extending substantially perpendicularly relative to and intersecting U-shaped rod-receiving passageway 1164 within bushing 1124.

Bushing 1124 defines an interior surface 1168, an exterior surface 1170, an upper end 1172a at the free ends of side walls 1160 and a bottom end 1172b at base 1158. Interior surface 1168 of base 1158 of bushing 1124 includes an inwardly angled ramp 1174 at bottom end 1172b of bushing 1124 that angles radially inwardly into internal cavity 1166 in a top to bottom direction, and an internal annular rib 1176 disposed on base 1158 and protruding radially inwardly into internal cavity 1166. Annular rib 1176 is longitudinally spaced from inwardly angled ramp 1174 a sufficient distance to enable capture of the full diameter portion of head 1112 of screw 1110 therebetween to, as detailed above, substantially inhibit longitudinal movement of head 1112 of screw 1110 relative to receiver 1120 while permitting angulation of head 1112 of screw 1110 relative to receiver 1120 (see FIGS. 11-13). Further, annular rib 1176 is sufficiently longitudinally spaced from U-shaped rod-receiving passageway 1164 to maintain a gap between head 1112 of screw 1110 and rod 400 (FIGS. 11-13) even where screw 1110 and/or rod 400 (FIGS. 11-13) are angled relative to one another and/or receiver 1120.

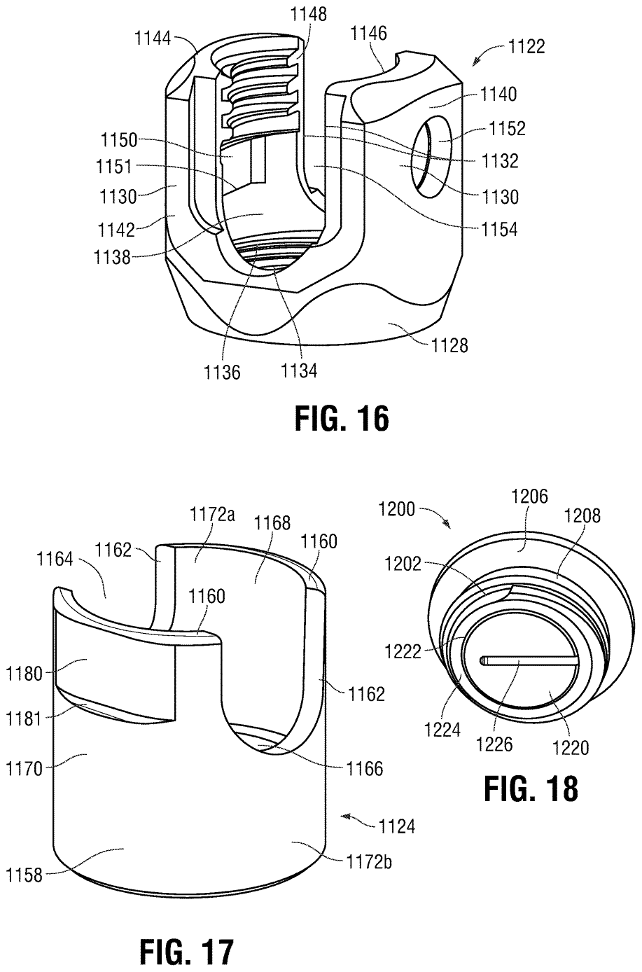

Bushing 1124 further includes anti-rotation surfaces 1180 positioned on side walls 1160 on the exterior surface 1170 of bushing 1124. Anti-rotation surfaces 1180 are complementary to anti-rotation surfaces 1150 (FIG. 16) and may be configured as recessed flats (as shown) recessed into exterior surface 1170 on each of side walls 1160, although other suitable anti-rotation surfaces are also contemplated. Anti-rotation surfaces 1180, more specifically, are configured to receive and mate with anti-rotation surfaces 1150 to rotationally lock shell 1122 and bushing 1124 relative to one another, thus inhibiting relative rotation therebetween. With anti-rotation surfaces 1150, 1180 rotationally locking shell 1122 and bushing 1124 relative to one another, saddles 1132, 1162 of shell 1122 and bushing 1124, respectively, are maintained in alignment with one another such that U-shaped rod-receiving passageways 1154, 1164 are maintained in alignment with one another.

Referring to FIGS. 14 and 15, end cap 1126 of receiver 1120 includes a neck 1182 and a head 1184. Neck 1182 defines a generally cylindrical configuration and includes threading 1186 disposed on an annular exterior surface thereof. Head 1184 defines a disc-shaped configuration having a diameter greater than a diameter of neck 1182 such that head 1184 protrudes radially outwardly farther than neck 1182 to define an annular shoulder 1188 therebetween.

End cap 1126 further defines a passageway 1190 extending longitudinally therethrough. An interior surface 1192 of end cap 1126 defining passageway 1190 includes an inwardly angled ramp 1194 disposed at the head end of end cap 1126.

Continuing with reference to FIGS. 14 and 15, to assemble receiver 1120, with anti-rotation surfaces 1150, 1180 aligned with one another, bushing 1124 is inserted into internal cavity 1134 of base 1128 of shell 1122 until anti-rotation surfaces 1150, 1180 mate with one another. In aspects, anti-rotation surfaces 1150, 1180 define shoulders 1151, 1181, respectively, configured to interact to define a stop to inhibit further insertion of bushing 1124 into receiver 1120 once anti-rotation surfaces 1150, 1180 mate with one another. Further, in this inserted position, saddles 1132, 1162 of shell 1122 and bushing 1124, respectively, and thus, U-shaped rod-receiving passageways 1154, 1164 of shell 1122 and bushing 1124, respectively, are maintained in alignment with one another.

Once bushing 1124 is inserted into shell 1122 as detailed above, neck 1182 of end cap 1126 is positioned in alignment with the annular space of internal cavity 1134 defined between base 1128 of shell 1122 and bushing 1124 and is rotated relative to shell 1122 to threadingly engage end cap 1126 with shell 1122 via engagement of threadings 1136, 1186 with one another. As an alternative to threaded engagement, other suitable mechanical engagements are also contemplated such as, for example, press-fitting, mechanical engagement, protrusion-aperture engagement, pin-slot engagement, or in any other suitable manner. Once the mechanical engagement is made, together therewith, or as an alternative to mechanical engagement, end cap 1126 may be fixedly secured to shell 1122 such as, for example, via welding, adhesion, etc. Thus, the above-detailed assembly may be performed during manufacture. Alternatively, assembly may be performed at the end-user (in aspects, without the fixed securement to enable assembly, disassembly, and re-assembly and/or to enable modularity for use of components of different materials, sizes, etc.).

With end cap 1126 engaged and/or secured to shell 1122, end cap 1126 maintains bushing 1124 within shell 1122. More specifically, inwardly angled ramp 1194 of end cap 1126 interfaces with inwardly angled ramp 1174 of bushing 1124 to inhibit passage of bushing 1124 through end cap 1126. Further, in this position, inwardly angled ramp 1174 of bushing 1124 covers inwardly angled ramp 1194 of end cap 1126 to inhibit contact between screw 1110 (FIGS. 11-13) and end cap 1126 in any angular orientation of screw 1110 (FIGS. 11-13) relative to end cap 1126.

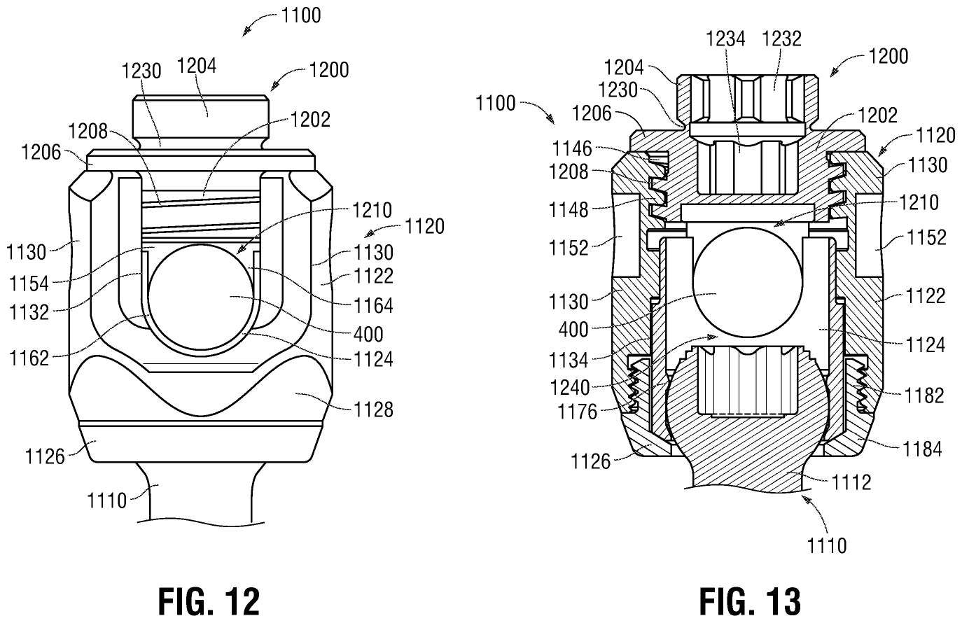

With additional reference to FIG. 12, with end cap 1126 engaged and/or secured to shell 1122, bushing 1124 is maintained in position relative to shell 1122 with saddles 1132, 1162 in alignment with one another and such that saddles 1162 of bushing 1124 are raised relative to saddles 1132 of shell 1122. In addition, saddles 1162 of bushing 1124 define smaller widths as compared to saddles 1132 of shell 1122. The raised and smaller width configurations of saddles 1162 of bushing 1124 relative to saddles 1132 of shell 1122 ensure that rod 400 extending through U-shaped rod-receiving passageways 1154, 1164 of shell 1122 and bushing 1124 contact saddles 1162 of bushing 1124 and are inhibited from contacting saddles 1132 of shell 1122. Thus, contact between metal (or relatively higher friction) components is inhibited.

Turning to FIGS. 11-13 and 18, set screw 1200 of screw assembly 1100 includes an engagement body 1202, a driver body 1204, and a collar 1206 disposed between and protruding radially outwardly from engagement body 1202 and driver body 1204. Engagement body 1202 includes external threading 1208 and is configured to threadingly engage arcuate threaded portions 1148 of side walls 1130 of shell 1122 to thereby close circular upper opening 1146 of shell 1122.

Upon sufficient engagement of engagement body 1202 within shell 1122, collar 1206 abuts free upper ends 1144 of side walls 1130 to define a stop to inhibit further advancement of engagement body 1202 into shell 1122. This stop, more specifically, defines the fully engaged position of set screw 1200 such that a gap 1210 is maintained between engagement body 1202 and rod 400 with rod 400 extending substantially coaxially through U-shaped rod-receiving passageways 1154, 1164. This gap 1210 facilitates sliding of rod 400 through and relative to receiver 1120 even with set screw 1200 fully engaged with shell 1122.

However, when rod 400 is angled relative to U-shaped rod-receiving passageways 1154, 1164, rod 400 may still potentially contact set screw 1200. Accordingly, set screw 1200 further includes a bushing disc 1220 that is configured to contact rod 400 when rod 400 is angled to thereby inhibit contact between rod 400 and other portions of set screw 1200. Whereas engagement body 1202, driver body 1204, and collar 1206 of end cap 1126 may be (monolithically) formed from strong material(s) such as any of the materials detailed above with respect to shell 122 (FIGS. 4-6B) or any other suitable material(s), e.g., a metal, bushing disc 1220 is formed from a low friction material such as any of the materials detailed above with respect to bushing 124 (FIGS. 4-6B), e.g., a polymeric material.

Bushing disc 1220 is partially received within a disc-shaped recess 1222 defined within a bottom face 1224 of engagement body 1202. Bushing disc 1220 defines a height greater than a height of disc-shaped recess 1222 such that bushing disc 1220 protrudes downwardly from bottom face 1224 of engagement body 1202. Bushing disc 1220 may be retained within disc-shaped recess 1222 via press-fitting or in any other suitable manner such as, for example via adhesion. In aspects, in order to facilitate press-fitting of bushing disc 1220 within disc-shaped recess 1222, disc-shaped recess 1222 defines a diameter equal to or less than a diameter of bushing disc 1220 and bushing disc 1220 includes a radial slit 1226 cut partially therethrough to facilitate compression of bushing disc 1220 for insertion into disc-shaped recess 1222 and such that, upon release, the expansion of bushing disc 1220 within disc-shaped recess 1222 facilitates the press-fit retention of bushing disc 1220 within disc-shaped recess 1222.

Driver body 1204 of set screw 1200 is coupled to collar 1206 via a transition 1230. In aspects, transition 1230 defines a pre-determined break point wherein transition 1230 breaks to thereby separate driver body 1204 from the remainder of set screw 1200. For example, as force (e.g., torque) applied to driver body 1204 reaches the pre-determined break point, transition 1230 breaks, thereby separating driver body 1204 from the remainder of set screw 1200. Drive body 1204 includes a cavity 1232 extending longitudinally through at least a portion of a length thereof and having a suitable geometric, e.g., hexagonal, star-shaped, etc., cross-section configured to facilitate engagement with an assembly tool (not shown), e.g., a rotational driver, to facilitate threaded engagement of engagement body 1202 within shell 1122, as detailed above. The rotational driver may provide the torque input to driver body 1204 to ultimately reach the pre-determined break point for separating driver body 1204 from the remainder of set screw 1200. More specifically, the rotational driver may be engaged (in fixed rotational orientation) within cavity 1232 to facilitate rotational driving of engagement body 1202 into engagement within shell 1122 to the fully engaged position at which point the pre-determined break point is reached to separate driver body 1204 from the remainder of set screw 1200. Set screw 1200 may further include an underlying cavity 1234 defined within engagement portion 1202 and exposed upon removal of driver body 1204 to enable receipt of an assembly tool (not shown), e.g., a rotational driver, for removal of set screw 1200. In aspects, the breakaway feature is omitted. In aspects, driver body 1204 is omitted and cavity 1234 of engagement portion 1202 is utilized for engaging and disengaging set screw 1200. Other set screw configurations are also contemplated.

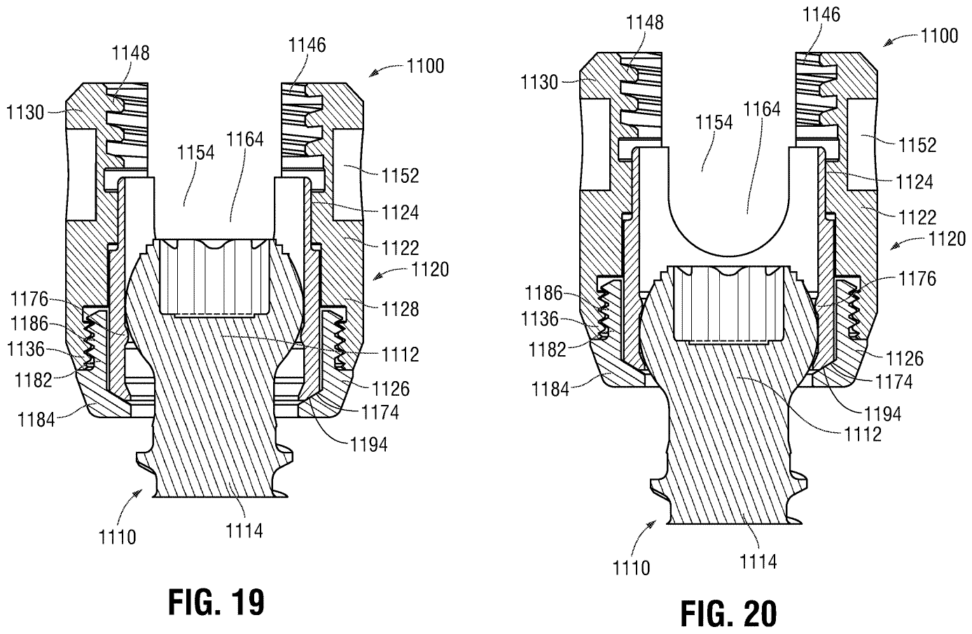

With reference to FIGS. 19 and 20, in conjunction with FIGS. 11-13, assembly and use of screw assembly 1100 is detailed. Initially, screw 1110, led by shank 1114, is inserted through opening 1146 of shell 1122 and through cavity 1166 of bushing 1124 through shell 1122, bushing 1124, and end cap 1126 such that shank 1114 of screw 1110 extends distally from receiver 1120 while head 1112 of screw 1110 is retained within receiver 1120. More specifically, as head 1112 of screw 1110 is urged longitudinally through receiver 1120, head 1112 contacts annular rib 1176 and compresses annular rib 1176 at least partially into base 1158 of bushing 1124 to enable passage of the full diameter portion of head 1112 through the reduced-diameter space defined by annular rib 1176 and into position between annular rib 1176 and inwardly angled ramp 1194 of bushing 1124. Once the full diameter portion of head 1112 traverses annular rib 1176, annular rib 1176 resiliently returns to its at-rest configuration to thereby capture head 1112 between annular rib 1176 and angled ramp 1194 while still permitting angulation of screw 1110 relative to receiver 1120, similarly as detailed above with respect to screw assembly 100 (FIGS. 7-10). Further, annular rib 1176 and saddles 1162 of bushing 1124 maintain a gap 1240 (FIG. 13) between head 1112 of screw 1110 and rod 400 when rod 400 is received within receiver 1120 (see FIG. 12), thus inhibiting contact therebetween regardless of the angulation of screw 1110 relative to receiver 1120 and rod 400 and facilitating sliding of rod 400 through receiver 1120.

Once screw 1110 is operably coupled to receiver 1120 as detailed above, screw 1110 may be anchored within tissue, e.g., vertebral bone, at a target location, rod 400 may be slidably received or longitudinally dopped into receiver 1120, and set screw 1200 may be secured to receiver 1120 to retain rod 400 in slidable engagement with screw assembly 1100. Thus, for example, sliding of rod 400 and angulation of screw 1110 are enabled, e.g., to adapt to growth of a child patient, while metal fretting and debris generation from metal-on-metal and/or other high-friction contacts are inhibited.

Aspects of this disclosure may be further described by reference to the following numbered paragraphs:
1. A receiver configured to operably couple a spinal screw with a spinal rod, the receiver comprising: a shell defining an internal cavity, a proximal opening in communication with the internal cavity, a distal opening in communication with the internal cavity, and first and second lateral openings in communication with the internal cavity, the proximal opening configured to receive a screw distally therethrough with a head of the screw disposed within the internal cavity and a shank of the screw extending distally through the distal opening, the first and second lateral openings configured to receive a rod extending through the internal cavity and laterally from opposing sides of the shell, wherein the shell is formed from a first material; and a bushing lining the first lateral opening, the second lateral opening, and at least a portion of the internal cavity, wherein the bushing is formed from a second material different from the first material, the bushing configured to inhibit contact between the shell and the screw, the shell and the rod, and the screw and the rod.
2. The receiver according to paragraph 1, wherein the first material is a metal, and the second material is a plastic.
3. The receiver according to paragraph 1 or 2, wherein the bushing includes a bushing body that lines the at least a portion of the internal cavity, a first ring that lines the first lateral opening, and a second ring that lines the second lateral opening.
4. The receiver according to any one of paragraphs 1-3, wherein the shell includes a first inwardly angled ramp adjacent to the distal opening, and wherein the bushing includes a second inwardly angled ramp lining the first inwardly angled ramp, the second inwardly angled ramp inhibiting contact between the screw and the shell without extending into the distal opening of the shell.
5. The receiver according to any one of paragraphs 1-4, wherein the bushing includes an annular rib protruding radially inwardly into an interior of the bushing, the annular rib configured to releasably retain the head of the screw within the internal cavity of the shell in spaced relation relative to the rod.
6. The receiver according to any one of paragraphs 1-5, wherein the bushing is configured to permit at least one of: sliding of the rod relative to the bushing and the shell; or angulation of the screw relative to the bushing and the shell.
7. A spinal screw assembly configured to operably couple a spinal screw with a spinal rod, the assembly comprising: a screw including a head and a shank extending from the head; and a receiver, including: a shell defining an internal cavity having a screw head-receiving portion and a rod-receiving portion, a proximal opening in communication with the internal cavity, a distal opening in communication with the internal cavity, and first and second lateral openings in communication with the internal cavity, the proximal opening configured to receive the screw distally therethrough with the head of the screw disposed within the screw head-receiving portion of the internal cavity and the shank of the screw extending distally through the distal opening, the first and second lateral openings configured to receive a rod extending through the rod-receiving portion of the internal cavity, wherein the shell is formed from a first material; and a bushing lining at least a portion of the internal cavity, the bushing formed from a second material different from the first material, the bushing configured to inhibit contact between the shell and the screw and to maintain a gap between the screw and the rod within the internal cavity, thereby inhibiting contact between the screw and the rod within the internal cavity.
8. The assembly according to paragraph 7, wherein the bushing lines the first and second lateral openings and is configured to inhibit contact between the shell and the rod.
9. The assembly according to paragraph 7 or 8, wherein the bushing includes an annular rib protruding radially inwardly into an interior of the bushing, the annular rib configured to releasably retain the head of the screw within the screw head-receiving portion of the internal cavity and to maintain the gap.
10. The assembly according to any one of paragraphs 7-9, wherein the receiver is configured to permit angulation of the screw relative to the receiver.
11. The assembly according to any one of paragraphs 7-10, wherein the first material is a metal, and the second material is a plastic.
12. The assembly according to any one of paragraphs 7-11, wherein the bushing includes a bushing body that lines the at least a portion of the internal cavity, a first ring that lines the first lateral opening, and a second ring that lines the second lateral opening.
13. The assembly according to any one of paragraphs 7-12, wherein the shell includes a first inwardly angled ramp adjacent to the distal opening, and wherein the bushing includes a second inwardly angled ramp lining the first inwardly angled ramp, the second inwardly angled ramp inhibiting contact between the screw and the shell without extending into the distal opening of the shell.
14. A spinal stabilization system, comprising: a rod; a screw including a head and a shank extending from the head; and a receiver, including: a shell defining an internal cavity having a screw head-receiving portion and a rod-receiving portion, a proximal opening in communication with the internal cavity, a distal opening in communication with the internal cavity, and first and second lateral openings in communication with the internal cavity, the proximal opening configured to receive the screw distally therethrough with the head of the screw disposed within rod-receiving portion and the shank of the screw extending distally through the distal opening, the first and second lateral openings configured to receive the rod extending through the internal cavity; and a bushing lining at least a portion of the interior surface of the shell and the first and second lateral openings, the bushing configured to permit sliding of the rod relative to the bushing and the shell, permit angulation of the screw relative to the bushing and the shell, inhibit contact between the shell and the screw, inhibit contact between the shell and the rod, and maintain a gap between the screw and the rod within the internal cavity.
15. The system according to paragraph 14, wherein the bushing includes a bushing body that lines the at least a portion of the internal cavity, a first ring that lines the first lateral opening, and a second ring that lines the second lateral opening.
16. The system according to paragraph 14 or 15, wherein the bushing includes an annular rib protruding radially inwardly into an interior of the bushing, the annular rib configured to releasably retain the head of the screw within the internal cavity and to maintain the gap between the screw and the rod within the internal cavity.
17. The system according to any one of paragraphs 14-16, wherein shell is formed from a first material, wherein the bushing is formed from a second material, and wherein the second material is relatively lower friction material than the first material.
18. The system according to paragraph 17, wherein the first material is a metal, and the second material is a plastic.
19. The system according to any one of paragraphs 14-18, wherein the bushing fully surrounds the rod within each of the first and second lateral openings of the shell.
20. The system according to any one of paragraphs 14-19, wherein the bushing includes an inwardly angled ramp configured to inhibit contact between the screw and the shell without extending into the distal opening of the shell.
21. A receiver configured to operably couple a spinal screw with a spinal rod, the receiver comprising: a shell including a base and first and second opposing side walls extending upwardly from the base, the shell defining a rod receiving passageway extending transversely through the shell; a bushing disposed within the shell and including a base and first and second opposing side walls extending upwardly from the base, the bushing defining a rod receiving passageway extending transversely through the bushing, wherein the rod receiving passageways of the shell and the bushing are aligned and configured to slidably receive a rod therethrough with the bushing supporting the rod to inhibit contact between the rod and the shell; and an end cap engaged to the base of the shell to retain the bushing within the shell.
22. The receiver according to paragraph 21, wherein the shell is formed from a first material and wherein the bushing is formed from a second material different from the first material.
23. The receiver according to paragraph 21 or 22, wherein the bushing and the shell define complementary anti-rotation surfaces configured to maintain the rod receiving passageways of the shell and the bushing in alignment with one another.
24. The receiver according to any one of paragraphs 21-23, wherein the bushing and the shell engage one another to inhibit upward movement of the bushing within the shell and wherein the bushing and the end cap engage one another to inhibit downward movement of the bushing within the shell.
25. The receiver according to any one of paragraphs 21-24, wherein the end cap is at least one of threadingly engaged with the base of the shell or welded to the base of the shell.
26. The receiver according to any one of paragraphs 21-25, further comprising a set screw configured to engage the first and second opposing side walls of the shell to retain the rod within the rod receiving passageways of the shell and the bushing, the set screw including a body and a bushing disc engaged with the body, wherein the bushing disc is configured to inhibit contact between the body of the set screw and the rod.
27. The receiver according to any one of paragraphs 21-26, wherein the bushing includes an annular rib protruding radially inwardly into an interior of the bushing, the annular rib configured to releasably retain a head of a screw within an internal cavity of the bushing in spaced relation relative to the rod.
28. A spinal screw assembly configured to operably couple a spinal screw with a spinal rod, the assembly comprising: a screw including a head and a shank extending from the head; and a receiver, including: a shell including a base and first and second opposing side walls extending upwardly from the base, the shell defining a rod receiving passageway extending transversely through the shell; and a bushing disposed within the shell and including a base and first and second opposing side walls extending upwardly from the base, the base defining an internal cavity configured to retain the head of the screw to inhibit contact between the screw and the shell, the first and second opposing side walls of the bushing defining a rod receiving passageway of the bushing extending transversely through the bushing, wherein the rod receiving passageways of the shell and the bushing are aligned and configured to slidably receive a rod therethrough with the bushing supporting the rod to inhibit contact between the rod and the shell.
29. The spinal screw assembly according to paragraph 28, wherein the shell is formed from a first material and wherein the bushing is formed from a second material different from the first material.
30. The spinal screw assembly according to paragraph 28 or 29, wherein the bushing and the shell define complementary anti-rotation surfaces configured to maintain the rod receiving passageways of the shell and the bushing in alignment with one another.
31. The spinal screw assembly according to any one of paragraphs 28-30, further comprising a set screw configured to engage the first and second opposing side walls of the shell to retain a rod within the rod receiving passageways of the shell and the bushing.
32. The spinal screw assembly according to paragraph 31, wherein the set screw includes a body and a bushing disc engaged with the body, wherein the bushing disc is configured to inhibit contact between the body of the set screw and the rod.
33. The spinal screw assembly according to any one of paragraphs 28-33, wherein the bushing includes an annular rib protruding radially inwardly into an interior of the bushing, the annular rib configured to maintain spacing between the head of the screw and the rod.
34. A spinal stabilization system, comprising: a rod; a screw including a head and a shank extending from the head; and a receiver, including: a shell including a base and first and second opposing side walls extending upwardly from the base, the shell defining a rod receiving passageway extending transversely through the shell; and a bushing disposed within the shell and including a base and first and second opposing side walls extending upwardly from the base, the base configured to retain the head of the screw to inhibit contact between the screw and the shell, the first and second opposing side walls of the bushing defining a rod receiving passageway of the bushing extending transversely through the bushing, wherein the rod is configured for slidable receipt within the rod receiving passageways of the shell and the bushing with the rod supported by the bushing to inhibit contact between the rod and the shell.
35. The system according to paragraph 34, wherein the bushing and the shell define complementary anti-rotation surfaces configured to maintain the rod receiving passageways of the shell and the bushing in alignment with one another.
36. The system according to paragraph 34 or 35, wherein the receiver further comprises a set screw configured to engage the first and second opposing side walls of the shell to retain the rod within the rod receiving passageways of the shell and the bushing.
37. The system according to paragraph 36, wherein the set screw includes a body and a bushing disc engaged with the body, wherein the bushing disc is configured to inhibit contact between the body of the set screw and the rod.
38. The system according to paragraph 37, wherein, in a fully engaged position of the set screw, a gap is defined between the bushing disc and the rod to enable sliding of the rod through the rod receiving passageways of the shell and the bushing.
39. The system according to paragraph 38, wherein the fully engaged position is defined by contact between a collar of the set screw and free ends of the first and second side walls of the shell.
40. The system according to any one of paragraphs 34-39, wherein the bushing is configured to maintain a gap between the rod and the head of the screw.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

1-15. (canceled)

16. A receiver configured to operably couple a spinal screw with a spinal rod, the receiver comprising:
a shell defining an internal cavity, a proximal opening in communication with the internal cavity, a distal opening in communication with the internal cavity, and first and second lateral openings in communication with the internal cavity, the proximal opening configured to receive a screw distally therethrough with a head of the screw disposed within the internal cavity and a shank of the screw extending distally through the distal opening, the first and second lateral openings configured to receive a rod extending through the internal cavity and laterally from opposing sides of the shell, wherein the shell is formed from a first material; and
a bushing lining the first lateral opening, the second lateral opening, and at least a portion of the internal cavity, wherein the bushing is formed from a second material different from the first material, the bushing configured to inhibit contact between the shell and the screw, the shell and the rod, and the screw and the rod.

17. The receiver according to claim 16, wherein the first material is a metal, and the second material is a plastic.

18. The receiver according to claim 16, wherein the bushing includes a bushing body that lines the at least a portion of the internal cavity, a first ring that lines the first lateral opening, and a second ring that lines the second lateral opening.

19. The receiver according to claim 16, wherein the shell includes a first inwardly angled ramp adjacent to the distal opening, and wherein the bushing includes a second inwardly angled ramp lining the first inwardly angled ramp, the second inwardly angled ramp inhibiting contact between the screw and the shell without extending into the distal opening of the shell.

20. The receiver according to claim 16, wherein the bushing includes an annular rib protruding radially inwardly into an interior of the bushing, the annular rib configured to releasably retain the head of the screw within the internal cavity of the shell in spaced relation relative to the rod.

21. The receiver according to claim 16, wherein the bushing is configured to permit at least one of: sliding of the rod relative to the bushing and the shell; or angulation of the screw relative to the bushing and the shell.

22. A spinal screw assembly configured to operably couple a spinal screw with a spinal rod, the assembly comprising:
a screw including a head and a shank extending from the head; and
a receiver, including:
a shell defining an internal cavity having a screw head-receiving portion and a rod-receiving portion, a proximal opening in communication with the internal cavity, a distal opening in communication with the internal cavity, and first and second lateral openings in communication with the internal cavity, the proximal opening configured to receive the screw distally therethrough with the head of the screw disposed within the screw head-receiving portion of the internal cavity and the shank of the screw extending distally through the distal opening, the first and second lateral openings configured to receive a rod extending through the rod-receiving portion of the internal cavity, wherein the shell is formed from a first material; and
a bushing lining at least a portion of the internal cavity, the bushing formed from a second material different from the first material, the bushing configured to inhibit contact between the shell and the screw and to maintain a gap between the screw and the rod within the internal cavity, thereby inhibiting contact between the screw and the rod within the internal cavity.

23. The assembly according to claim 22, wherein the bushing lines the first and second lateral openings and is configured to inhibit contact between the shell and the rod.

24. The assembly according to claim 22, wherein the bushing includes an annular rib protruding radially inwardly into an interior of the bushing, the annular rib configured to releasably retain the head of the screw within the screw head-receiving portion of the internal cavity and to maintain the gap.

25. The assembly according to claim 22, wherein the receiver is configured to permit angulation of the screw relative to the receiver.

26. The assembly according to claim 22, wherein the first material is a metal, and the second material is a plastic.

27. The assembly according to claim 22, wherein the bushing includes a bushing body that lines the at least a portion of the internal cavity, a first ring that lines the first lateral opening, and a second ring that lines the second lateral opening.

28. The assembly according to claim 22, wherein the shell includes a first inwardly angled ramp adjacent to the distal opening, and wherein the bushing includes a second inwardly angled ramp lining the first inwardly angled ramp, the second inwardly angled ramp inhibiting contact between the screw and the shell without extending into the distal opening of the shell.

29. A spinal stabilization system, comprising:
a rod;
a screw including a head and a shank extending from the head; and
a receiver, including:
a shell defining an internal cavity having a screw head-receiving portion and a rod-receiving portion, a proximal opening in communication with the internal cavity, a distal opening in communication with the internal cavity, and first and second lateral openings in communication with the internal cavity, the proximal opening configured to receive the screw distally therethrough with the head of the screw disposed within rod-receiving portion and the shank of the screw extending distally through the distal opening, the first and second lateral openings configured to receive the rod extending through the internal cavity; and
a bushing lining at least a portion of the interior surface of the shell and the first and second lateral openings, the bushing configured to permit sliding of the rod relative to the bushing and the shell, permit angulation of the screw relative to the bushing and the shell, inhibit contact between the shell and the screw, inhibit contact between the shell and the rod, and maintain a gap between the screw and the rod within the internal cavity.

30. The system according to claim 29, wherein the bushing includes a bushing body that lines the at least a portion of the internal cavity, a first ring that lines the first lateral opening, and a second ring that lines the second lateral opening.

31. The system according to claim 29, wherein the bushing includes an annular rib protruding radially inwardly into an interior of the bushing, the annular rib configured to releasably retain the head of the screw within the internal cavity and to maintain the gap between the screw and the rod within the internal cavity.

32. The system according to claim 29, wherein shell is formed from a first material, wherein the bushing is formed from a second material, and wherein the second material is relatively lower friction material than the first material.

33. The system according to claim 32, wherein the first material is a metal, and the second material is a plastic.

34. The system according to claim 29, wherein the bushing fully surrounds the rod within each of the first and second lateral openings of the shell.

35. The system according to claim 29, wherein the bushing includes an inwardly angled ramp configured to inhibit contact between the screw and the shell without extending into the distal opening of the shell.
